(19) United States
(12) Patent Application Publication
(10) Pub. No.: US 2024/0102998 A1
(43) Pub. Date: Mar. 28, 2024

(54) METHODS AND MATERIALS FOR IMPROVED IMMUNOASSAYS USING SYNTHETIC ANTIGENS

(71) Applicant: THE TERASAKI INNOVATION CENTER, Glendale, CA (US)

(72) Inventors: Neng Jen Remi Shih, Winnetka, CA (US); Thoa Nong, Granada Hills, CA (US); Jar-How Lee, Los Angeles, CA (US)

(21) Appl. No.: 18/264,013

(22) PCT Filed: Feb. 2, 2022
(86) PCT No.: PCT/US22/14947

§ 371 Date: Aug. 2, 2023

Related U.S. Application Data
(60) Provisional application No. 63/248,049, filed on Sep. 24, 2021.
(60) Provisional application No. 63/145,055, filed on Feb. 3, 2021.

(51) Int. Cl. G01N 33/543; G01N 33/564

(52) U.S. Cl. CPC G01N 33/54393; G01N 33/564

(57) ABSTRACT

Described herein are materials and methods for improved immunoassays using synthetic antigens in which samples are treated to bind antibodies specific to histidine polymer antigens.

### CROSS-REFERENCE TO RELATED APPLICATIONS

This application is a national phase entry under 35 USC § 371 of International Application PCT/US22/14947, filed Feb. 2, 2022, which claims the benefit and priority of U.S. Patent Application Nos. 63/248,049 filed Sep. 24, 2021 and 63/145,055 filed Feb. 3, 2021, which are incorporated herein by reference in their entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “56379B_SeqListing.xml; Size: 32,356 bytes; Created: Aug. 1, 2023”. The subject matter of the Sequence Listing is incorporated herein in its entirety by reference.

### FIELD OF THE INVENTION

The present invention is directed to materials and methods for conducting improved immunoassays using synthetic antigens.

### BACKGROUND

Immunoassays using protein antigens which specifically bind with specific antibodies are well known in the art for detecting the presence of such antibodies in biological samples. The general process relies upon the ability of antibodies to specifically bind with specific “target” antigens in a detectable manner with a variety of means being used to indicate the specific binding such as by producing a detectable signal. The binding assays can be performed according to various formats including competitive and non-competitive assays and can use detectable labels include enzymatic, radioactive, fluorogenic and other elements and molecules and other means well known to the art can be used to detect such binding. Not only can antigens be used to detect the presence of particular antibodies in test samples but antibodies can also be used to detect the presence of antigens in test samples.

Immunoassays are widely used for detecting antibodies associated with infectious disease pathogens including but not limited to Hepatitis, HIV, Lyme disease, syphilis, rotavirus and more recently the SARS-CoV-2 virus associated with COVID-19. Such assays can be used not just to determine the presence of infection but also for the determination of vaccination against disease pathogens e.g., detection of Hepatitis B antibody in blood for the detection of infection of Hepatitis B or vaccination against Hepatitis B (https://www.hepb.org/prevention-and-diagnosis/diagnosis/hbv-blood-tests/). Immunoassays are also known for determining the compatibility of transplanted organ and tissue by the detection of antibodies to the highly polymorphic Human leukocyte antigens (HLA) which are a type of target molecule within a transplanted organ to which the recipient's immune system responds resulting in transplant rejection. Detection of antibodies to other antigens including those implicated in Xenotransplantation of non-human animal tissues such as Swine leukocyte antigens (SLA) are also contemplated.

The accuracy of immunoassays is of critical importance, particularly when their results affect medical care such as when the assay is used to determine whether the test subject is infected by a transmissible disease. Immunoassays can fail in either or both of two manners. False-negative results for the presence of antibodies to a pathogen can also indicate the absence of immunity in a subject previously infected with a disease or immunized against such a pathogen. In the context of HLA tissue compatibility testing, a false-negative test for specific antibodies can inappropriately indicate tissue compatibility between tissue donors and recipients with serious negative consequences. Immunoassay testing can also report a false-negative result in which they fail to detect the presence of an analyte which is actually present in a test sample. Such false-negative results can cause a delay in care needed by a test subject but can also falsely indicate that the test subject does not have an infectious disease with the result that the subject fails to implement isolation precautions that would prevent spread of the disease to others.

False-positive results are those in which the assay falsely indicates the presence of an antibody specific for a particular antigen (or conversely an antigen specific for a particular antibody). The occurrence of false positive reactions are also undesirable because they can falsely indicate that a test subject suffers from a health condition and result in the administration of unneeded and potentially harmful treatment for a condition which does not exist. In cases of transmissible disease, a false positive result can cause undesirable anxiety in the test subject and their loved ones but also result in their inappropriate undesired and ineffectual isolation. In cases of HLA tissue compatibility testing false positive results indicating an incompatibility between donor and recipient tissues can exclude what could otherwise have been lifesaving transplantation matches.

Of interest to the present application is poly-histidine tagging which is well known for the purification of recombinant proteins via affinity purification. Specifically, DNA sequences coding for a string of six to nine histidine residues are attached to the DNA encoding a desired recombinant protein. The expressed His-tagged fusion protein can then be purified by exposure to various type of immobilized metal ions as well as by anti-His-tag antibodies.

Also of interest to the present invention is the disclosure of Bengtsson et al., Arthritis & Rheumatism, Vol. 39, 1654-1663 (1996) which discloses the occurrence of antibodies specific for poly(amino acids) including poly-histidine in patients with systemic lupus erythematosus (SLE).

False-negative and false-positive reactions can occur for a variety of reasons not all of which are fully understood. Thus, there remains a need in the art for improved methods of conducting immuno-assays which reduce the occurrence of false positive or false negative reactions.

### SUMMARY

The present invention is related to the observation that individuals in the population frequently produce antibodies to proteins including poly-histidine amino acid sequences, especially in auto-immune disease patients, such as systemic lupus erythematosus (SLE). Recombinant protein therapeutics produced using poly-histidine tags can also induce antibody production in subjects to whom they are administered. In some cases the antibodies are specific for the poly-histidine sequences and for others the antibodies are specific for epitopes comprising fusion proteins of naturally occurring polypeptide sequences with two or more histidine peptides at their C-terminal ends.

In addition, poly-histidine tagged recombinant proteins are used in many antibody screening assays for human or animal pathogens, such as those for SARS-CoV-2 Spike or Nucleocapsid proteins. Thus, naturally produced anti-poly-histidine antibodies produced in human and other animal subjects will react with poly-histidine tags being used on the SARS-CoV-2 recombinant protein targets in many COVID-19 antibody assays naturally produced poly-histidine antibodies will interfere with the COVID-19 antibody assays will provide false-positive results in those assays by binding to the poly-His epitope of the recombinant SARS-CoV-2 Spike or Nucleocapsid target proteins. Anti-poly-histidine antibodies can also cause higher noise in negative samples, raise the threshold for the positive/negative cut-off and decrease assay sensitivity thus producing false negative results. The risk of such false positive or false negative results is not limited to testing for SARS-CoV-2 protein antibodies but also exists for any immunoassay for the detection of antibodies in which the target protein was recombinantly produced using poly-histidine tags thus causing potential risks to public health.

Similarly, tissue transplant compatibility screening (such as Human Leukocyte Antigen (HLA) screening) typically uses panels of synthetic antigens to which donor or recipient serum is administered to detect cross-reactions between antibodies present in the serum sample and the synthetic antigens. Such synthetic antigens are frequently produced by recombinant means in which the protein antigen is a fusion protein which includes a poly-histidine-tag. Alternatively, synthetic antigens can be produced by well-known chemical synthetic methods such as by the phosphoramidite method in which poly-histidine residues are incorporated into the chemically synthesized oligo- or polynucleotide structure.

Anti-poly-histidine antibodies present in the biological sample can then create false positive reactions in the synthetic antigen screening assays for cross-reactive antibodies such as used in HLA assays for tissue transplantation matching.

The invention provides methods of reducing the presence and/or effects of such anti-poly-histidine antibodies in the biological fluids including, blood, serum and plasma for antibody testing using His-Tag recombinant proteins by detecting the presence of such anti-poly-histidine antibodies or treating biological samples in which antibodies reactive for poly-histidine antigens are present to block, absorb or otherwise remove them prior to exposure to synthetic antigen panels.

A further aspect of the invention relates to the observation that individuals in the population also produce antibodies specific for fusion proteins of naturally occurring polypeptide sequences and fused with two or more histidine peptides on their C-terminal ends. Such fusion proteins can include those used in diagnostic tests such as for HIV, Lyme disease, syphilis, rotavirus SARS including SARS-CoV-2, HLA markers and are not limited to those such as HLA-A, HLA-B, HLA-C, DRA and DQA1. These antibodies have the potential to provide false positive results for the presence of antibodies directed to the naturally occurring proteins when the assay uses synthetic versions of the subject proteins created with poly-histidine tails.

According to one aspect of the invention a method is provided for screening a biological sample for antibodies that specifically bind a target antigen by the steps of obtaining a biological sample from a subject, subjecting the biological sample to poly-histidine polymers in order to block or remove anti-poly-histidine antibodies from the biological sample, contacting the target antigen with the biological sample, and detecting binding of antibodies in said biological sample to the target antigen. According to one aspect of the invention the poly-histidine polymer comprises a fusion protein of a recombinant protein with a poly-histidine sequence of at least two but preferably six contiguous histidine peptides. The recombinant protein can comprise the entire natural sequence of a naturally occurring protein or a C-terminal truncated version of the naturally occurring target antigen protein having at least two contiguous histidine peptides at its C-terminal end. In some cases, where the poly-histidine polymer comprises a truncated version of a target antigen protein having at least two contiguous histidine peptides at its C-terminal end the polymer is capable of blocking or removing antibodies specific for antigenic epitopes comprising only 1) poly-histidine sequences or 2) fusion proteins of a protein with contiguous histidine peptides on its end but not 3) the protein not having contiguous peptides on its end. In this manner antibodies specific only for the protein corresponding to the target protein but not having histidine peptides on its C-terminal end will not be blocked or removed from an antigen-antibody assay.

According to one embodiment the target antigen can be a recombinantly produced peptide including a poly-histidine sequence of at least six contiguous histidine peptides. According to a further alternative the target antigen is a synthetic antigen including a poly-histidine sequence of at least six contiguous histidine peptides. Alternatively the target antigen does not include a poly-histidine sequence of at least six contiguous histidine peptides.

The sample can be any biological sample from a human or other animal and can include those from the group including but not limited to tissues and biological fluids including blood, plasma, serum, urine, saliva and the like.

Proteins can frequently comprise multiple antigenic epitopes to which different antibodies have binding specificity and the present invention is related to the recognition that antibodies can be reactive to an epitope presented by a histidine polymer present on a recombinant protein while different antibodies are specific for other antigenic epitopes on the same protein. The target antigen can thus include those containing histidine polymers comprising poly-histidine amino acid sequences. The target antigen can be a synthetic antigen which can be synthesized by recombinant or chemical synthetic means such as by the use of the phosphoramidite method. According to one aspect of the invention the target antigen is a recombinantly produced polypeptide including a histidine polymer sequence of at least two or alternatively five or six or more contiguous histidine peptides.

Target antigens can be of any sort but preferred antigens include those associated with human or non-human animal pathogens including but not limited to those of the group consisting of HIV, Lyme disease, syphilis, rotavirus and SARS associated coronavirus including SARS-CoV-2. Testing for the presence of antibodies to such antigens frequently utilizes recombinantly produced synthetic antigens in which protein antigens are recombinantly produced using poly-histidine tags for purification purposes. Other antigens of interest include Human leukocyte antigens (HLA) used in tissue compatibility testing for transplantation.

Useful methods include those in which anti-poly-histidine antibodies present in biological samples are contacted with and blocked by or bound to poly-histidine polymer sequences can be carried out wherein the histidine polymers are present in solution or immobilized on solid supports including beads, microtiter wells, as well as on solid phase supports used in lateral flow devices in which a biological fluid flows from a first location to a second location along a solid phase substrate.

According to another aspect of the invention a method of screening for antibodies that specifically bind a target antigen is provided comprising preparing a panel comprising a solid-phase substrate having said target antigen immobilized or attached thereto; obtaining a biological sample from a subject, subjecting the biological sample to an affinity purification step in which it is contacted with histidine polymers immobilized on a solid substrate to remove anti-poly-histidine antibodies from the biological sample, contacting the panel with the biological sample, and detecting binding of antibodies in said biological sample to the target antigen.

Detection methods are provided for the presence of anti-poly-histidine antibodies in biological samples to determine whether human and non-human animal subjects have such antibodies. Such methods comprise of screening for antibodies that specifically bind a target antigen comprising preparing a panel comprising a solid-phase substrate having a target antigen immobilized thereon and at least one other antigen comprising a histidine polymer sequence comprising at least two or optionally at least six contiguous histidine peptides, obtaining a biological sample from a subject, contacting the panel with the serum sample, detecting binding of antibodies in said serum sample to the target antigen, and detecting binding of antibodies in said serum sample to the poly-histidine antigen.

In addition to synthetic antigen targets including poly-histidine being used for the detection of antibodies it should be recognized that antibodies themselves can constitute antigens and that synthetically produced antibodies which include antibodies and antibody fragments such as Fab fragments and scFv and the like can be produced using poly-histidine tags. Accordingly, such antibodies can themselves be the targets of anti-poly-histidine antibodies produced by human and non-human animal subjects and the presence of such naturally occurring anti-poly-histidine antibodies can bind to and interfere with synthetically produced antibodies. For example, in ELISA sandwich assays, if the capture antibody carries a poly-histidine tag, biological fluids containing anti-poly-histidine antibodies will react to the capture antibody and give false positive readings regardless of whether the target antigen has a poly-histidine tag or not. The blocking or removal of such anti-poly-histidine antibodies which can potentially bind with synthetically produced antibodies can thereby eliminate potential sources of interference and error.

Kits are provided for screening for antibodies that specifically bind to a target antigen and/or to a poly-histidine antigen comprising a panel of solid-phase substrates wherein the panel comprises a solid phase substrate having a target antigen immobilized thereon and at least one other antigen comprising at least two or more preferably six contiguous histidine peptides immobilized thereon. Alternatively, the histidine polymer comprises a fusion protein of a recombinant protein fusion proteins of a Human leukocyte antigen (HLA), and antigens selected from the group consisting of HIV, Lyme disease, syphilis, rotavirus and SARS associated coronavirus antigens with at least two contiguous histidine peptides with a poly-histidine sequence of at least two but preferably six contiguous histidine peptides.

Also provided are kits for blocking or removing antibodies that specifically bind to a poly-histidine antigen comprising an oligopeptide comprising at least two or six contiguous histidine peptides. According to one aspect of the invention a kit is provided comprising a solid-phase substrate wherein an oligopeptide comprising at least two or six contiguous histidine peptides is immobilized thereon.

Kits are also provided comprising: a solid phase substrate to which a target produced antigen is immobilized, a solution comprising histidine polymers having at least two and optionally six or more contiguous histidine peptides, and a component capable of preventing non-specific antibody binding. According to one aspect of the invention the target antigen is a recombinantly produced polypeptide including a poly-histidine sequence of at least two or more preferably six contiguous histidine peptides. Alternatively, the histidine polymer comprises a fusion protein of a naturally occurring polypeptide sequence or a C-terminal truncated version of the target antigen protein sequence with at least two contiguous histidine peptides or more preferably at least five contiguous histidine peptides. According to an further alternative aspect of the invention the target antigen does not include a poly-histidine sequence of at least two or five or six contiguous histidine.

Kits according to the invention can include various additional components which assist in the binding or detection of true positive reactions as well as blocking non-specific binding which components can include Bovine Serum Albumin (BSA), milk proteins or other components but most preferred is Fetal Calf Serum (FCS).

A further aspect of the invention relates to the observation that there exist sera which are reactive to a subset of proteins with a poly-histidine tag but which are not reactive to all His-tag recombinant proteins or to poly-histidine peptides. Thus, while many sera reactive to recombinant antigens with histidine tags can be removed by incubation with Histidine polymers there exist antibodies which react only to fusion peptides of naturally occurring polypeptide sequences but not to poly-histidine peptides alone. Accordingly, the histidine polymers used as specific binding targets and for blocking and affinity purification can include and fusion proteins selected from the group of consisting of fusion proteins of naturally occurring polypeptide sequences with at least two contiguous histidine peptides or more preferably at least five histidine peptides.

Thus, various approaches have been identified to either block or absorb the antibodies that react to the carboxyl-end of the recombinant protein and the poly-histidine tag by using a fusion protein that contain a portion of the carboxyl-end of the target protein and the poly-histidine tag. A universal polyhistidine peptide that contains a contiguous histidine sequence equal or longer than the His tag in that target protein can be used without verifying the minimum length of the histidine that form the epitope of the poly-histidine antibody. Multiple poly-histidine fusion proteins can be combined in a single mixture to absorb different poly-histidine antibody targets in the serum.

### BRIEF DESCRIPTION OF THE FIGURES

FIG. 1 depicts the antibody reactivity of sera on a COVID antibody assay after different absorption treatments;

FIG. 2 depicts absorption efficiency of treatment of anti-poly-histidine antibody containing serum with poly-histidine beads;

FIG. 3 depicts absorption efficiency of treatment of anti-poly-histidine antibody containing serum with poly-histidine beads;

FIGS. 4A-4D depict the effects of incubating serum containing anti-HLA antibodies with poly-histidine beads;

FIG. 5 depicts the effects of incubating serum containing anti-HLA antibodies on poly-histidine beads;

FIG. 6 depicts the effects of incubating serum containing anti-HLA antibodies on poly-histidine beads;

FIG. 7 depicts the ability of poly-histidine peptides of different lengths to block the anti-poly-histidine antibody interaction with SARS-CoV-2 antigens with histidine-tag in solution;

FIG. 8 depicts the effect of Fetal calf serum (FCS) and a histidine polymer on anti-poly-histidine antibody reactivity;

FIG. 9 depicts the effects of poly-histidine positive serum No. 3 in poly-histidine blocking;

FIG. 10 depicts the effects of poly-histidine positive serum No. 3 on various lengths of poly-histidine polymers;

FIG. 11 depicts the effects of poly-histidine positive serum No. 4 in poly-histidine blocking;

FIG. 12 depicts the effects of poly-histidine positive serum No. 4 on various lengths of poly-histidine polymers;

FIGS. 13A-13D depict the of effects of poly-histidine positive serum No. 2 on poly-histidine polymers; and

FIG. 14 depicts the of effects of poly-histidine positive serum No. 5 on poly-histidine polymers.

### DETAILED DESCRIPTION

The present invention relates to compositions and methods for reducing false positive antibody detection results by detecting and/or removing antibodies specific for poly-histidine peptide epitopes (histidine polymers) of recombinantly produced proteins prior to contacting the biological sample to target antigens selected for their reactivity to antibodies of interest.

Affinity purification methods for the binding and removal of antibodies specific for histidine polymers can be readily carried out according to methods well known to those of ordinary skill in the art. Suitable affinity resins for protein immobilization are well known to the art and are commercially available from sources such as ThermoFisher Scientific which rely on various linking technologies. Preferred histidine polymers for reaction with anti-poly-histidine antibodies include those comprising at least two or five or six contiguous histidine amino acid residues but can have longer stretches of histidine residues presenting reactive epitopes for such antibodies. The histidine polymers can be produced chemically by means well known to the art such as solid-phase peptide synthesis (SPPS) and other methods but are preferably produced by recombinant synthesis methods wherein the histidine polymers and synthetic antigens comprising the histidine polymers are produce by mammalian, bacterial, yeast or insect expression systems.

Preferred methods of binding peptide antigens including those with histidine polymers use specific functional groups to bind the antigen to an activated support whose reactive groups occur on spacer arms that are several atoms long. For large protein antigens, especially those with multiple sites of immobilization, the spacer arm length becomes less important since the antigen itself serves as an effective spacer between the support matrix and the epitope. Generally, if an antigen is cross-linked to a carrier protein to facilitate antibody production, best results are obtained when the antigen is immobilized for affinity purification using the same chemistry (e.g., reaction to primary amines, sulfhydryls, carboxylic acids or aldehydes). In this way, all epitopes will be available for antibody binding, allowing greater efficiency in purification and recovery of the specific immunoglobulin.

Binding and elution conditions for antigen-specific affinity purification of antibodies are generally very similar because they are based on native affinity interactions of antibodies with their respective antigens. Accordingly, typical affinity purification procedures use binding conditions that mimic physiologic pH and ionic strength. The most common binding buffers are phosphate-buffered saline (PBS) and Tris-buffered saline (TBS) at pH 7.2 and preferred incubation conditions are at temperatures from room temperature to 37° C. Suitable incubation times can readily be determined by those of ordinary skill in the art with time periods from 10 to 30 minutes being generally suitable. Once the antibody has been bound to an immobilized antigen, additional binding buffer is used to wash unbound material from the support. Wash buffers containing additional salt or detergent are used to disrupt any weak interactions to minimize nonspecific binding.

Specific, purified antibodies can be eluted from affinity resins by sufficiently altering the pH or ionic strength of the buffer to disrupt the antigen-binding interaction. Most antibodies tolerate wide ranges of pH without suffering inactivation with low pH being generally preferred for elution.

The solid-phase assays of the invention may be carried out on ELISA plates including 96 well ELISA trays, 384 well solid supports, other solid phase chips, membranes, filter paper and the like including with microparticles, microbeads, magnetic beads, beads or microspheres of any material, e.g. silica, gold, latex, polymers such as polystyrene, polysulfone and polyethyl, or hydrogel. Additional exemplary microparticles are encoded with the dyes and the antigens are immobilized to the encoded microparticles. The microparticles used in the methods of the invention are commercially available from sources such from Luminex Inc., Invitrogen (Carlsbad, CA), Polysciences Inc. (Warrington, PA) and Bangs Laboratories (Fishers, IN) to name a few.

The microparticles of the invention may comprise a detectable label or another identifying characteristic. The microparticles may comprise a single fluorescent dye or multiple fluorescent dyes. In one embodiment, the microparticles are internally labeled with fluorescent dyes and contain surface carboxyl groups for covalent attachment of biomolecules. In another embodiment, the microparticles are internally labeled with fluorescent dyes and contain a surface layer of Avidin for near covalent binding of biotin and biotinylated ligands. In another embodiment, the microparticles may comprise a combination of different dyes, such as a fluorescent and a non-fluorescent dye. For example, the microparticles may be labeled with E)-5-[-[2-methoxycarbonyl)ethenyl]cytidine, which is a nonfluorescent molecule, that when subjected to ultraviolet (UV) irradiation yields a single product, 3-β-D-ribofuranosyl-2,7-dioxopyrido[2,3-d]pyrimidine, which displays a strong fluorescence signal. In another embodiment, the microparticles may comprise bar codes as an identifiable characteristic as described in U.S. Patent Publication No. US 2007/0037195.

In another embodiment, the microparticles may be nanocrystals or quantum dots. These nanocrystals are substances that absorb photons of light, then re-emit photons at a different wavelength (fluorophores). In addition, additional florescent labels, or secondary antibodies may be conjugated to the nanocrystals. These nanocrystals are commercially available from sources such as Invitrogen and Evident Technologies (Troy, NY).

The methods and those described hereafter can also be performed wherein antibody binding is detected with a secondary antibody, preferably wherein the secondary antibody comprises a label selected from the group consisting of a radioactive label, fluorescent label, enzymatic label, colorimetric label avidin label and biotin label.

The invention can be carried out with any system that detects the identifiable characteristic or label, such as FLOW cytometry. Detection of fluorescent labels may also be carried out using a microscope or camera that will read the image on the microparticles, such as the Bioarray BeadChip (Bioarray Solutions, Ltd., Warren, NJ). The BeadChip format combines microparticle (“bead”) chemistry with semiconductor wafer processing in which binding to the microparticle is recorded using an optical microscope and camera.

The invention also may be carried out using column chromatography, affinity chromatography, thin layer chromatography, liquid-phase immunodiagnostic (LIPA) assays, liquid-phase chemiluminescent ELISA and liquid-phase immunoradiometric (IRMA), lateral flow strips and systems to name a few.

Biological samples includes whole blood, blood derivatives, red blood cell concentrates, plasma, serum, fresh frozen plasma, whole blood derived platelet concentrates, apheresis platelets, pooled platelets, intravenous gamma-globulin, cryoprecipitate, cerebrospinal fluid, tissues and cells such as epithelial cells, such as those collected from the buccal cavity, stem cells, leukocytes, neutrophils and granulocytes. The biological samples may be obtained from a human donor of tissue or cells intended for transplantation or a human donor of blood or blood derivatives intended for transfusion. The biological sample may be obtained from a healthy bone marrow donor or a subject of a paternity test. The biological sample may also be obtained from a human subject that is an intended recipient of a transplant or transfusion, or the human subject that is donating the tissue or organ intended for transplantation or transfusion. Alternatively, the biological sample may be obtained directly from tissues or cells that are intended for transplantation in a human recipient. In addition, the biological sample may be obtained from blood or blood derivatives that are intended for transfusion in a human recipient.

The immunoassays of the invention can use labels for detection of antigen-antibody binding including radioactive labels such as ³H, ¹⁴O, ³²P, ³⁵S, or ¹²⁵I. In addition, the labels may be a fluorescent or chemiluminescent compound, such as fluorescein isothiocyanate, phycoerythrin, rhodamine, or luciferin. The labels may be enzymes such as alkaline phosphatase, β-galactosidase, biotin and avidin or horseradish peroxidase (Bayer et al., Meth. Enz., 184:138-163 (1990)).

Specific binding of antibodies within a biological sample to antigens may be carried out using Western blot analysis with immunoblotting, immunocytochemistry, immunohistochemistry, dot blot analysis, flow cytometry, ELISA assays or RIA assays. These techniques and other approaches are conventional in the art (See Sambrook et al., Molecular Cloning: A Laboratory Manual, Cold Springs Harbor Laboratories (New York, 1989).

Of interest to the present invention are assay methods making use of flow cytometry. Wilson et al., J. Immunol. Methods 107: 231-237 (1988) disclose the use of polyacrylamide microspheres coupled with cell membrane proteins in immunofluorescence assays for antibodies to membrane-associated antigens. The method is said to make possible the rapid flow cytometric analysis of plasma membrane antigens from cell populations that would otherwise be unsuitable for use in flow cytometry. Scillian et al., Blood 73: 2041-2048 (1989) disclose the use of immunoreactive beads in flow cytometric assays for detection of antibodies to HIV. Frengen et al., Clin. Chem. 40/3: 420-425 (1994) disclose the use of flow cytometry for particle-based immunoassays of ce-fetoprotein (AFP). This reference further reports the ability of serum factors to cross-link labeled mouse monoclonal antibodies of irrelevant specificity to different particle types coated with various immunoglobulins.

Flow cytometry methods using lymphocytes are also known but suffer with difficulties because of the activity of auto-antibodies. See Shroyer et al., Transplantation 59:626-630. Moreover, when using flow cytometry with lymphocytes, use of ten or more different lymphocytes tends to result in confusing signals. As a consequence, studies using lymphocytes have been limited by presenting a small panel of HLA antigens that do not effectively simulate the distribution of HLA antigens in a normal human population.

The kits useful herein may further comprise any components necessary to carry out the detection assays that are conventional in the art. For example, the kits may comprise buffers, loading dyes, gels such as polyacrylamide gels and molecular weight markers for preparing SDS-PAGE gels to carry out Western blots. The kits may also comprise filters, membranes blocking buffers, control buffers, isotype control antibodies, wash buffers or buffers and reagents for detection to carry out immunoblotting or dot blotting analysis such as labeled secondary antibodies. The kit may also comprise fixing reagents, blocking buffers, control buffers, wash buffers, staining dyes and detection reagents including anti-idiospecific antibodies. Furthermore, the kits may comprise the necessary reagents and tools to carryout flow cytometry, ELISA assays, RIA assays or microtoxicity assays.

In some embodiments, the detecting step comprises detecting labeled ligand bound to the complex to determine the presence or absence of antibodies reactive against selected antigens. In some embodiments, detecting of the labeled ligand is carried out by flow cytometry. In some embodiments, the detecting step comprises detecting the presence of the complex using a solid phase immunoassay or a multiplexed bead immunoassay.

The solid-phase substrate can be any solid substrate known in the art. In some embodiments, the solid-phase substrate is selected from the group consisting of microparticle, microbead, magnetic bead, ion torrent bead, flow cytometer bead and an affinity purification column. In some embodiments, the solid-phase substrate is a microbead. In some embodiments, the microbead is a latex microbead. The microbead, in some embodiments, has a diameter ranging from about 2 μm to about 15 μm, inclusive. Microbeads having a diameter of about 2 μm, 3 μm, 4 μm, 5 μm, 6 μm, 7 μm, 8 μm, 9 μm, 10 μm, 11 μm, 12 μm, 13 μm, 14 μm or 15 μm are also contemplated.

In some embodiments, each solid phase substrate is detectably distinguishable from other solid phase substrates within the composition. In some embodiments, the detectably distinguishable solid phase substrates are distinguishable by fluorescent labels.

Other aspects and advantages of the present invention will be understood upon consideration of the following illustrative examples. Two sera that have anti-poly-histidine antibodies and HLA Class II DR antibodies and convalescent serum from a COVID-19 recovered patient were absorbed with different amounts of poly-histidine beads, then tested on COVID Plus kit (One Lambda) and HLA Class II Single Antigen Kit (One Lambda). The results showed the absorption with poly-histidine beads has no affect in the true positive antibodies but significantly reduced the anti-poly-histidine antibodies. It further revealed the true HLA antibodies when the anti-poly-histidine antibody was removed.

Aspects of the invention are further disclosed by the examples provided below. In Example 1 absorption of anti-poly-histidine antibodies from human sera reduced unexpected reactivities of the sera to recombinantly produced SARS-CoV-2 antigens with a His-tag. In Example 2 absorption of anti-poly-histidine antibodies from human sera reduced unexpected reactivities of the sera to recombinantly produced HLA antigens expressed with a histidine-tag. In Example 3 free poly-histidine peptides were used to block anti-poly-histidine antibodies. In Example 4 different lengths of poly-histidine peptides were tested for their ability to block the anti-poly-histidine antibody interaction with SARS-CoV-2 antigens with histidine-tag in the solution. In Example 5 histidine polymer was used to block anti-poly-histidine antibodies present in serum and prevent their reaction with recombinantly produced SARs-CoV-2 antigens expressed with poly-histidine tags and used in a commercially available antibody assay. In Example 6 sera having antibodies specific for fusion proteins of C-terminal HLA alleles with poly-histidine tags were tested for binding specificity to poly-histidine polymer and fusion proteins of different C-terminal HLA alleles with poly-histidine tags. In Example 7 different fusion proteins with poly-histidine tags were tested for their ability to remove antibody activity of a serum sample. In Example 8, fusion proteins with different numbers of poly-histidine residues were tested for their ability to block a poly-histidine positive serum. In Example 9 a serum was tested against various fusion proteins having different numbers of poly-histidine residues. In Example 10, different fusion proteins with poly-histidine tags were tested for their ability to remove antibody activity of a serum sample. In Example 11 a serum was tested against various fusion proteins having different numbers of poly-histidine residues. In Example 12 different fusion proteins with poly-histidine tags were tested for their ability to remove antibody activity of a serum sample. In Example 13, different fusion proteins with poly-histidine tags were tested for their ability to remove antibody activity of a serum sample. In Example 14, different fusion proteins with poly-histidine tags were tested for their ability to remove antibody activity of a serum sample.

### Example 1

According to this example, polystyrene beads were first conjugated with poly-histidine, then used to absorb anti-poly-histidine antibodies in the serum. Approximately 2,000,000 Polystyrene beads were first conjugated with 100 μg of poly-L-histidine (Sigma-Aldrich P9386) and resuspended in 150 μl of final storage buffer.
1. Different volumes of bead suspension were first pelleted in microcentrifuge tubes
2. Supernatant was removed
3. Serum (treated with 1 ul of 0.2M EDTA per 20 μl serum) was added to the pelleted beads, vortexed to resuspend the beads
4. Incubate at room temperature for 10 minutes.
5. Centrifuge to pellet the beads.
6. Serum (supernatant) is carefully removed from the tube and diluted 1:10 in PBS for the assay.

Two sera containing anti-poly-histidine antibodies, a convalescent serum from a COVID-19 recovered patient and a negative control serum were absorbed with equivalent of 5 μl, 10 μl, and 5 μl twice of poly-histidine beads, then tested on LABScreen™ COVID Plus assay (One Lambda). The SARS-CoV-2 antigens used in the LABScreen™ COVID Plus assay contain a His-Tag at the C-terminal of the protein. The poly-histidine conjugated beads were also included in the assay.

The manufacturer recommended protocol for LABScreen™ COVID Plus assay was used and is briefly described here:
1. 20 μl of treated serum was mixed with 5 μl of COVID Plus microbeads and incubated for 30 minutes at room temperature,
2. the microbeads were washed 3× with wash buffer, then
3. incubated with phycoerythrin (PE) conjugated goat anti-human IgG for 30 minutes.
4. The microbeads were spun 1× and washed 2× with wash buffer and analyzed on a Luminex FM3D analyzer (Luminex) according to the manufacturer's instructions.

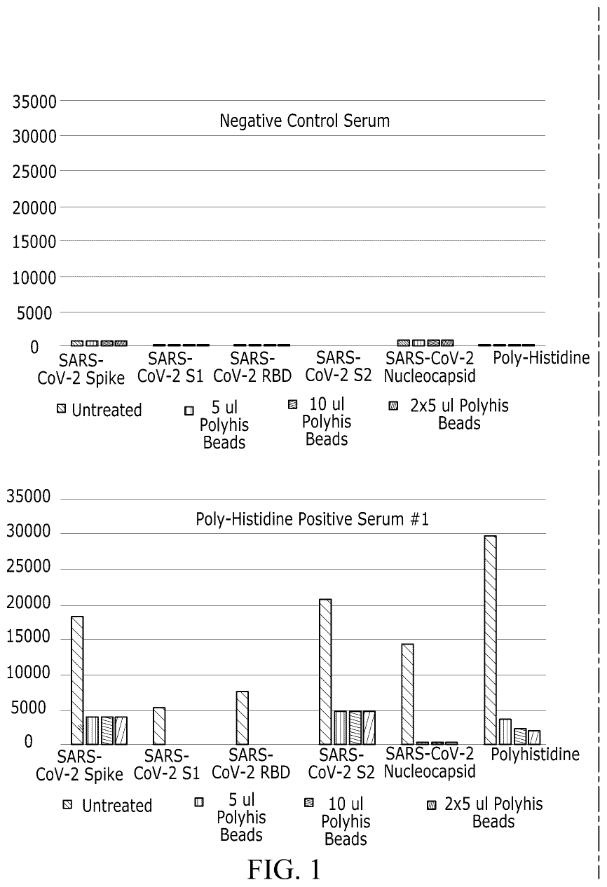

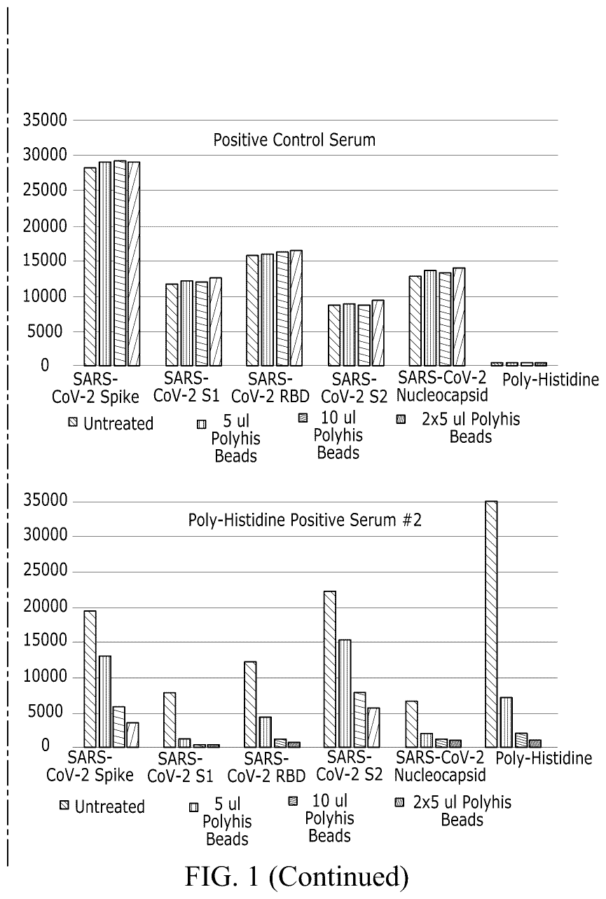

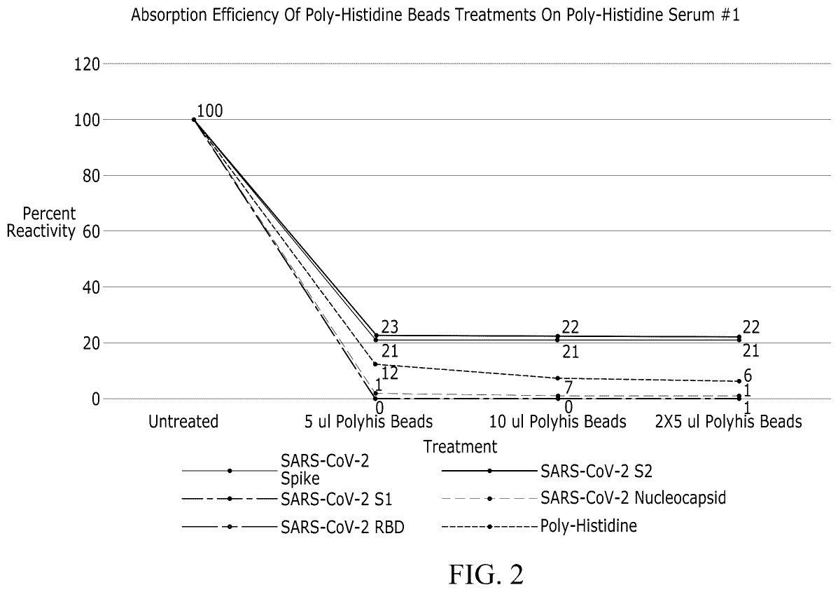

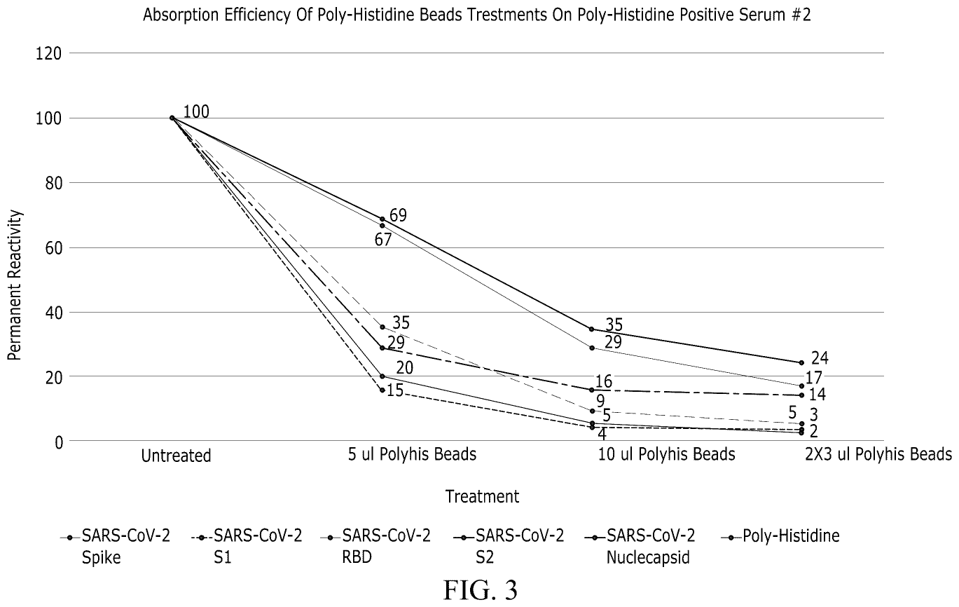

The results from the assay showed the absorption with poly-histidine beads has no effect on the reactivities of the true positive SARS-CoV-2 antibodies but reduced the reactivity to His-Tag on the SARS-CoV-2 antigens between 76%-99% from the anti-poly-histidine antibodies (FIG. 1-3).

### Example 2

According to this example, polystyrene beads were first conjugated with poly-histidine and used to absorb anti-poly-histidine antibodies in the serum. Approximately 2,000,000 Polystyrene beads were first conjugated with 100 μg of poly-L-histidine (Sigma-Aldrich P9386) and resuspended in 150 μl of final storage buffer.
1. Different volumes of bead suspension were first pelleted in microcentrifuge tubes
2. Supernatant was removed.
3. Serum (treated with 1 ul of 0.2M EDTA per 20 μl serum) was added to the pelleted beads, vortexed to resuspend the beads.
4. Incubate at room temperature for 10 minutes.
5. Centrifuge to pellet the beads.
6. Serum (supernatant) is carefully removed from the tube for the assay.

Two sera that have anti-poly-histidine antibodies, an HLA Class II positive serum and a negative control serum were absorbed with equivalent of 5 μl, 10 μl, and 5 μl twice of poly-histidine beads, then tested on LABScreen™ Single Antigen assay (LS2A01, One Lambda). The HLA DR antigens used in LS2A01 contain a His-Tag at the C-terminal of the protein.

The manufacturer recommended protocol for LABScreen™ Single Antigen assay was used and is briefly described here:
1. 20 μl of treated serum was mixed with 5 μl of COVID Plus microbeads and incubated for 30 minutes at room temperature,
2. the microbeads were washed 3× with wash buffer, then
3. incubated with phycoerythrin (PE) conjugated goat anti-human IgG for 30 minutes.
4. The microbeads were spun 1× and washed 2× with wash buffer and analyzed on a Luminex FM3D analyzer (Luminex) according to the manufacturer's instructions.

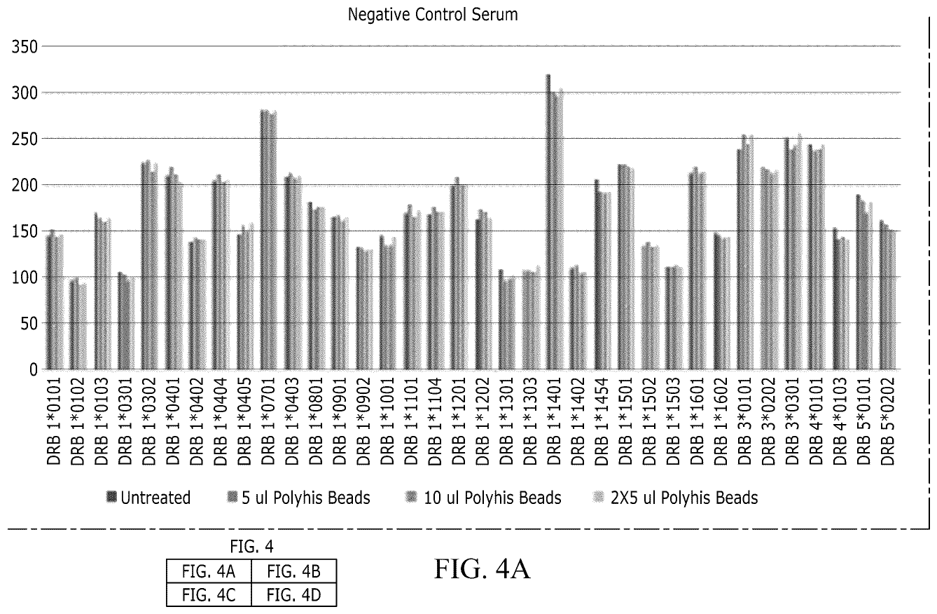

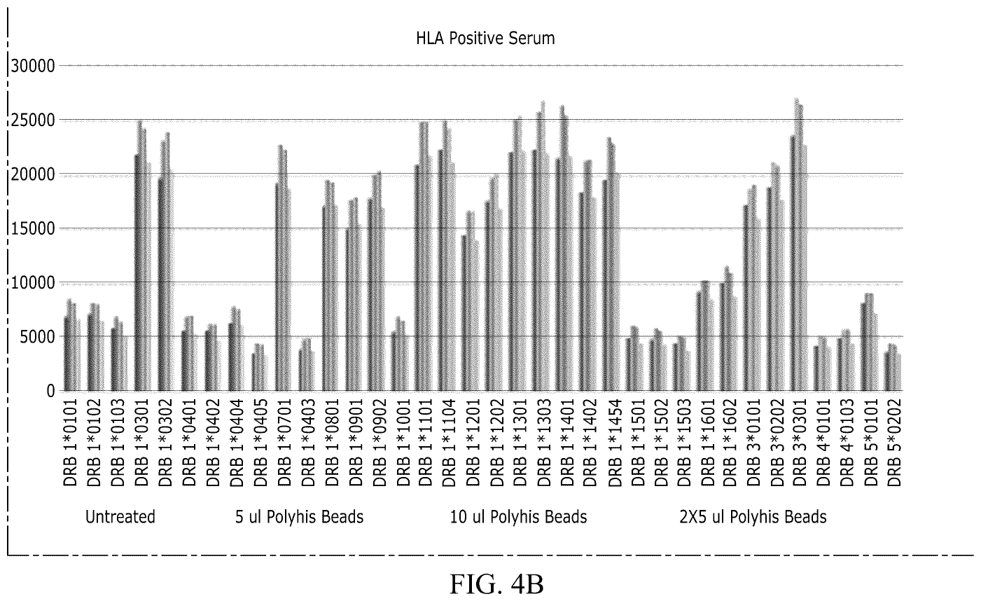

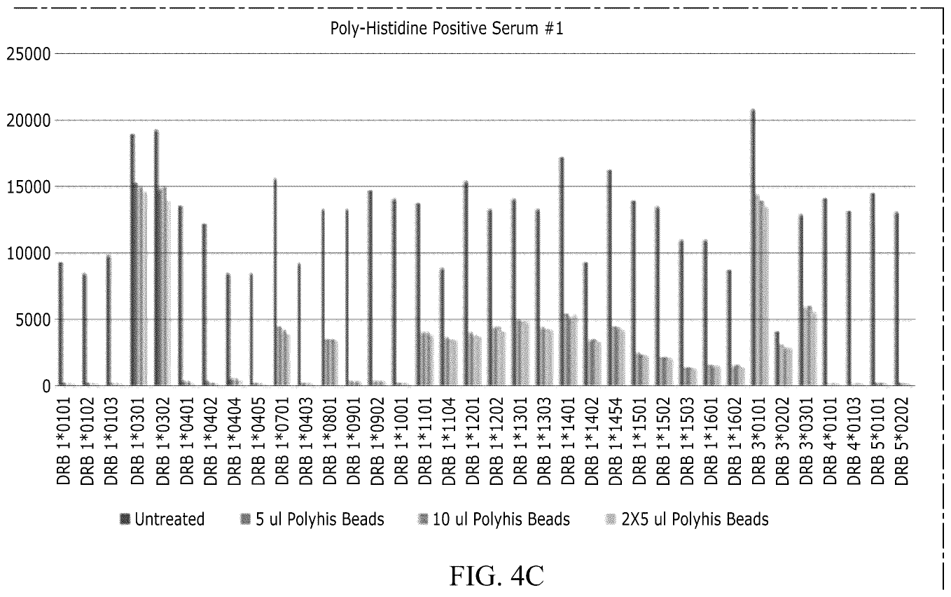

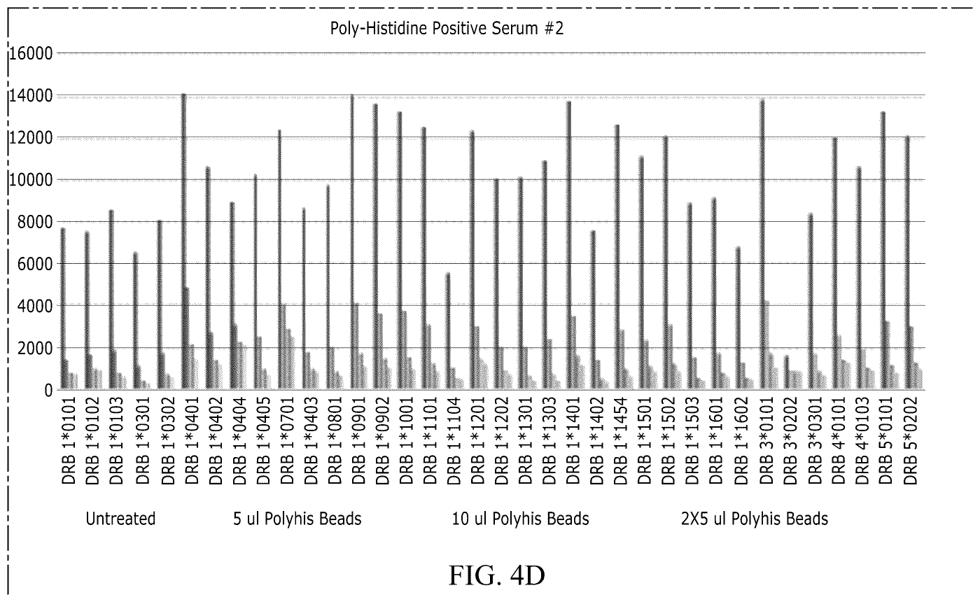

The results from the assay showed the absorption with poly-histidine beads has no effect on the reactivities of the true HLA positive antibodies but has reduced the reactivity to histidine-Tag on the HLA-DR antigens revealed the true HLA antibodies in poly-histidine Positive Serum #1 when the anti-poly-histidine antibody was removed (FIG. 4).

### Example 3

According to this example, histidine polymers were tested for their ability to block the interaction of anti-poly-histidine antibodies in solution with recombinant produced HLA-DR antigens with histidine-tag.

Two (2) μg of poly-histidine (Sigma-Aldrich) with a Molecular weight average of 7600 (55 histidine) was added to 20 μl of poly-histidine positive serum #1 and incubated for 10 minutes at room temperature, then spun at 7,000 RPM for 5 minutes to remove the aggregates.

The clarified serum was then tested on LABScreen™ Class II Single Antigen assay (LS2A01) as described in Example 1.

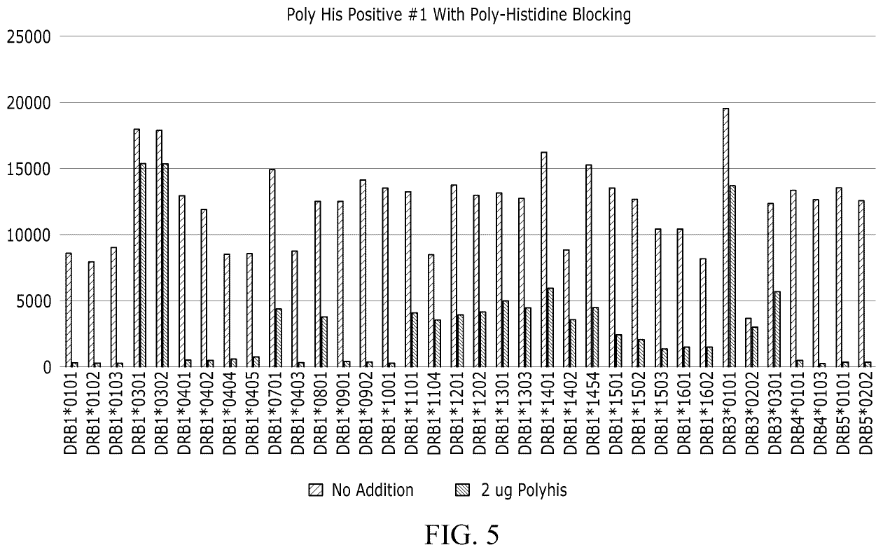

The reactivity of anti-poly-histidine antibody in poly-histidine positive serum #1 after poly-histidine treatment demonstrate the treatment has removed the anti-poly-histidine antibody, but retained the HLA-DR antibody reactivity in the serum (FIG. 5).

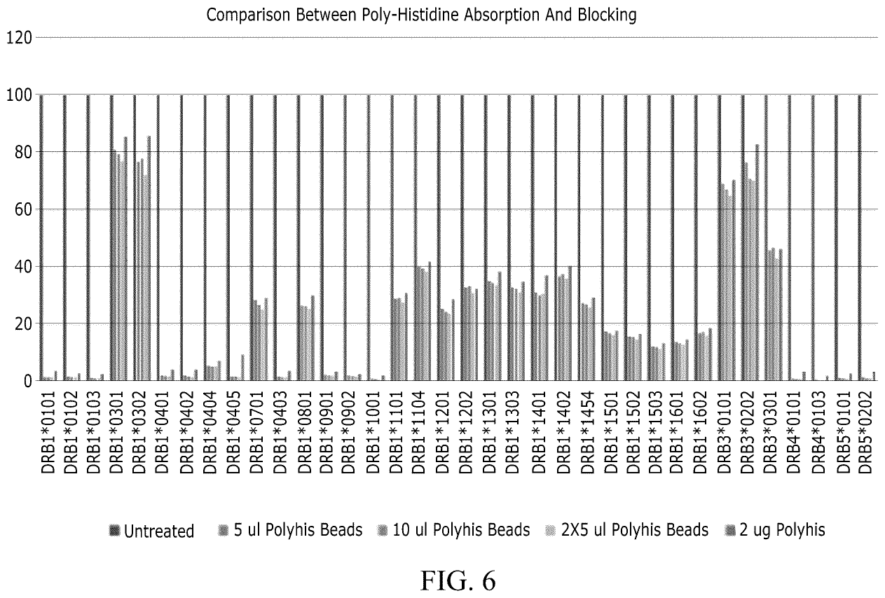

The blocking efficiency of each HLA antigen was calculated as the percentage of residual reactivity to the reactivity of the untreated serum and compared to the blocking efficiencies of poly-histidine absorption from Example 2. The results showed that using 2 μg of poly-histidine achieved comparable efficiency of removing anti-poly-histidine antibody reactivity in the serum by bead absorption (FIG. 6).

### Example 4

According to this example, different lengths of poly-histidine peptides were tested for their ability to block the anti-poly-histidine antibody interaction with SARS-CoV-2 antigens with histidine-tag in the solution.

Two (2) μg of either a 55-mer (Sigma Aldrich) or a 6-mer (Abcam) poly-histidine were added to 20 μl of poly-histidine positive serum #1 and incubated for 10 minutes at room temperature, then was either spun at 7,000 RPM for 5 minutes to remove the aggregates or without centrifugation.

These processed serum samples were then tested on LABScreen™ COVID Plus assay (One Lambda). The poly-histidine conjugated beads were also included in the assay.

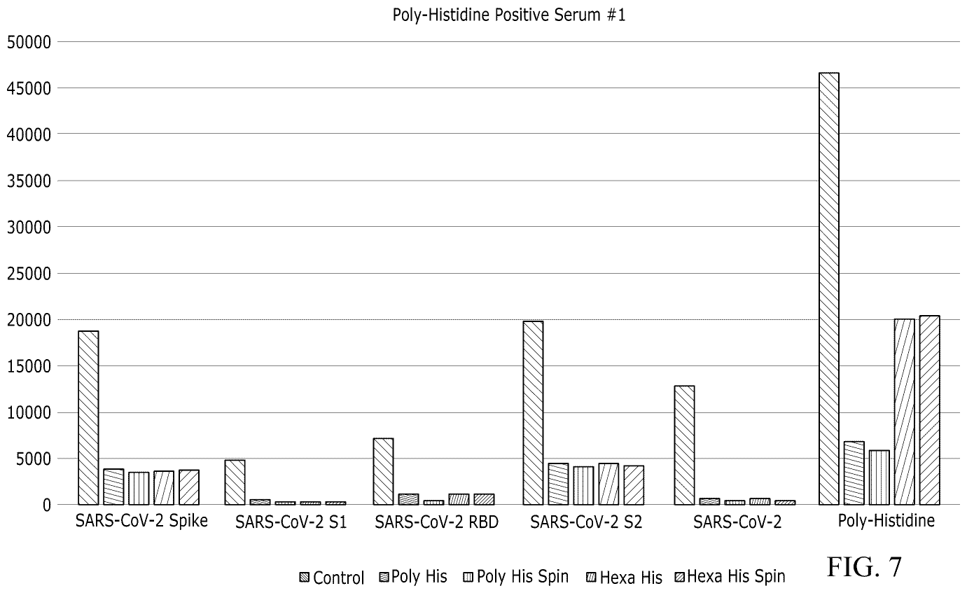

Both 2 μg Poly-histidine (55-mers) and 2 μg Hexa-histidine (6-mer) were able to block the anti-poly-histidine antibody reactivity in the serum samples as shown in FIG. 7.

### Example 5

According to this example, poly-histidine peptide was added into Fetal calf serum (FCS) at 2 μg/μl and the cocktail was used for blocking anti-poly-histidine antibody in the Poly-Histidine Positive Serum #1. 20 μl of serum was treated with 1 μl of the FCS cocktail, incubate at 37° C. for 30 minutes and then diluted 1:10 for the COVID Plus assay.

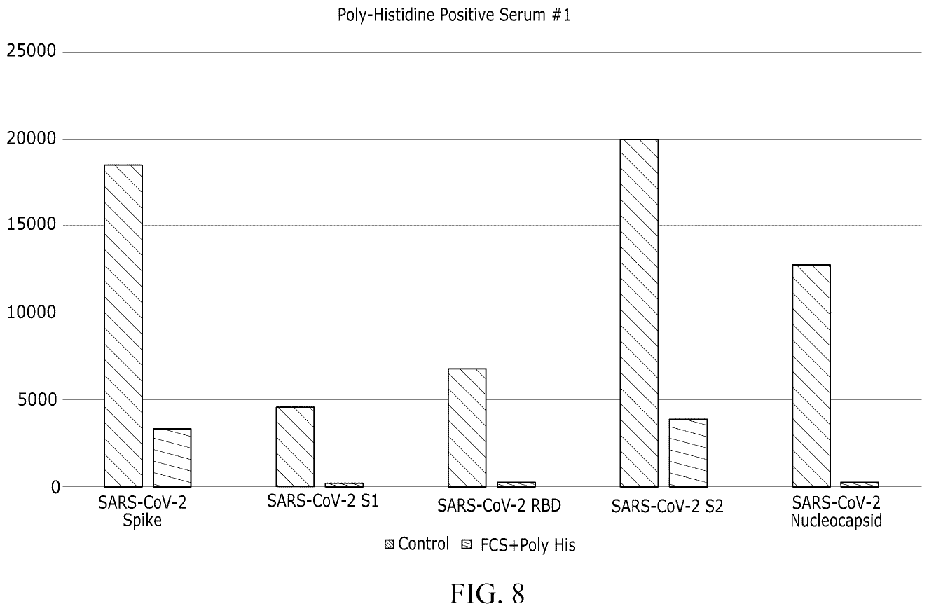

The results in FIG. 8 showed the FCS cocktail treatment is sufficient to block the anti poly-histidine antibody reactivity in the serum.

### Example 6

A further aspect of the invention relates to the observation that there exist sera which are reactive to a subset of proteins with a poly-histidine tag but which are not reactive to all His-tag recombinant proteins or to poly-histidine peptides. Thus, while many sera reactive to recombinant antigens with His tags can be removed by incubation with Histidine polymers there exist antibodies which react only to fusion peptides of DQ antigens with His tags, or fusion peptides of DR antigens with His tags, or fusion peptides of HLA-C peptides with His tags but not to poly-histidine peptides alone. In particular, three sera have been identified and are described below. One serum, hereinafter poly-histidine positive Serum No. 3 reacts to only the DQ antigens with a His tag. Another serum, hereinafter poly-histidine positive Serum No. 4 reacts only to the Cw antigens with a His tag and a further serum, hereinafter poly-histidine positive serum No. 5 that reacts to only the DR antigens with a His tag.

TABLE 1

| | | Antibody | | | |
|---|---|---|---|---|---|
| | C terminal with poly- | | DR-poly- | DQ-poly- | C-poly- |
| Allele | histidine tag | Poly-His | His | His | His |
| DRA*0101H | KGL RKSNAAERRG PL LEHHHHHH | + | + | − | − |
| [SEQ ID NO: 1] | | | | | |
| DQA1*0101H | RGL RSVGASRHQG PL LEHHHHHH | + | - | + | − |
| [SEQ ID NO: 2] | | | | | |
| C*01:02H | NSA QGSDESLIAC KA LEHHHHHH | + | − | - | + |
| [SEQ ID NO: 3] | | | | | |
| Poly-His | HHHHHH | + | − | − | - |
| [SEQ ID NO: 4] | | | | | |

The epitope of the DQ specific serum was found to include the amino acid at the C′-end of the DQA1, the two amino acid linker and at least 2 histidine of the His tag.

The epitope of the Cw specific serum was found to include the amino acid at the C′-end of the Cw, the two amino acid linker and at least 5 histidine of the His tag.

Fusion proteins for the cytoplasmic potion of HLA-A, B, C, DRA1 and DQA1 and various length of Histidine were produced in E. coli and were used to characterize the antibody epitopes on Western blot and to demonstrate the utility of removing His tag reactive antibody in the sera.

According to this example, fusion proteins with various lengths of poly-histidine residues were prepared and were tested for reactivity to anti-poly-histidine antibodies.

Specifically, DNA corresponding HLA Cytoplasmic (CT) region with various histidine residues was fused to the C terminus of GST carrier protein after a 21 amino acid linker, SD(GGGGS)x3-GGGG (SEQ ID NO: 25), at the end of GST molecule. The GST-HLA CT proteins with various histidine residues are expressed by an E. coli T7 Expression system, pET28a, in BL21(DE3). Bacteria cells harbor the pET28a with the corresponding GST-HLA CT constructs were induced by 1 mM IPTG at 37° C., on a platform shaker at 200 rpm, for 6 hours. Cell pellets were harvested and stored at −20° C.

TABLE 2

| Locus | Cytoplasmic Domain |
|---|---|
| DRA1-LE-Hx6 | KGLRKSNAAERRGPLLEHHHHHH |
| SEQ ID NO: 5] | |
| DQA1-LE-Hx6 | RGLRSVGASRHQGPLLEHHHHHH |
| [SEQ ID NO: 6] | |
| DQA1-LE-Hx5 | RGLRSVGASRHQGPLLEHHHHH |
| [SEQ ID NO: 7] | |
| DQA1-LE-Hx4 | RGLRSVGASRHQGPLLEHHHH |
| [SEQ ID NO: 8] | |
| DQA1-LE-Hx3 | RGLRSVGASRHQGPLLEHHH |
| [SEQ ID NO: 9] | |
| DQA1-LE-Hx2 | RGLRSVGASRHQGPLLEHH |
| [SEQ ID NO: 10] | |
| DQA1-LE-Hx1 | RGLRSVGASRHQGPLLEH |
| [SEQ ID NO: 11] | |
| DQA1-LE | RGLRSVGASRHQGPLLE |
| [SEQ ID NO: 12] | |
| DQA1 | RGLRSVGASRHQGPL |
| [SEQ ID NO: 13] | |
| A-LE-Hx6 | VLTACKVLEHHHHHH |
| [SEQ ID NO: 14] | |
| B-LE-Hx6 | VLT...ALEHHHHHH |
| [SEQ ID NO: 15] | |
| C-LE-Hx6 | SLIACKALEHHHHHH |
| [SEQ ID NO: 16] | |
| C-LE-Hx5 | SLIACKALEHHHHH |
| [SEQ ID NO: 17] | |
| C-LE-Hx4 | SLIACKALEHHHH |
| [SEQ ID NO: 18] | |
| C-LE-Hx3 | SLIACKALEHHH |
| [SEQ ID NO: 19] | |
| C-LE-Hx2 | SLIACKALEHH |
| [SEQ ID NO: 20] | |
| C-LE-Hx1 | SLIACKALEH |
| [SEQ ID NO: 21] | |
| C-LE | SLIACKALE |
| [SEQ ID NO: 22] | |
| C | SLIACKA |
| [SEQ ID NO: 23] | |

HLA Cytoplasmic Tail Fusion Purification.

GST-HLA CT proteins with variable histidine residues were purified by Glutathione Sepharose 4B columns. In brief, frozen cell pellets were lysed by BugBuster® (primary amine-free) Extraction Reagent (Millipore) in the presence of Benzonase® Nuclease (Millipore) for 20 min at room temp. Clear bacterial lysates were obtained by centrifugation and were loaded into Glutathione Sepharose 4B (Cytiva) columns equilibrated with 1×PBS. After washing extensively with 1×PBS, GST-HLA CT proteins with variable histidine residues were eluted with 10 mM Glutathione (reduced) in 1×PBS.

Characterization and antibody blocking of poly-histidine positive Serum No. 3.

One microgram of each purified fusion protein from the following list was loaded on the 4-20% Tris-Glycine polyacrylamide gel and western blotted with 20 μl of poly-histidine positive serum No. 3.
1. GST-DRA1-LE-Hx6
2. GST-DQA1-LE-Hx6
3. GST-DQA1-LE-Hx5
4. GST-DQA1-LE-Hx4
5. GST-DQA1-LE-Hx3
6. GST-DQA1-LE-Hx2
7. GST-DQA1-LE-Hx1
8. GST-DQA1-LE
9. GST-DQA1

According to these results poly-histidine positive Serum No. 3 reacts to only DQA1-LE fusion proteins with at least 2 Histidine (Samples 1-6).

### Example 7

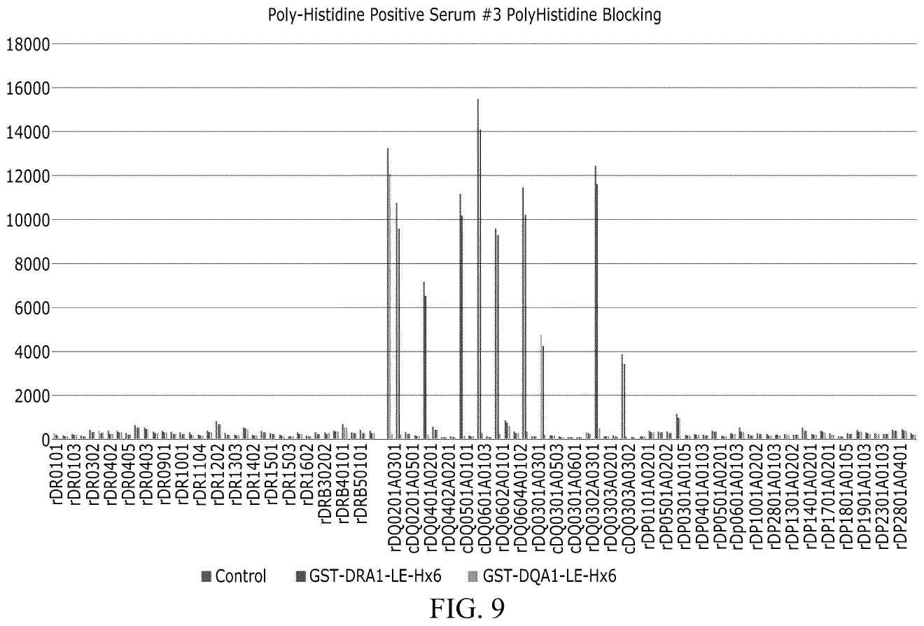

According to this example, twenty μl of poly-histidine positive Serum No. 3 was preincubated with 5 μg of either GST-DRA1-LE-Hx6 or GST-DQA1-LE-Hx6, then tested on LS2A01 Class II single antigen panel. The results shown in FIG. 9 demonstrate GST-DQA1-LE-Hx6 removed the antibody reactivity completely while GST-DRA1-LE-Hx6 didn't remove the antibody reactivity.

### Example 8

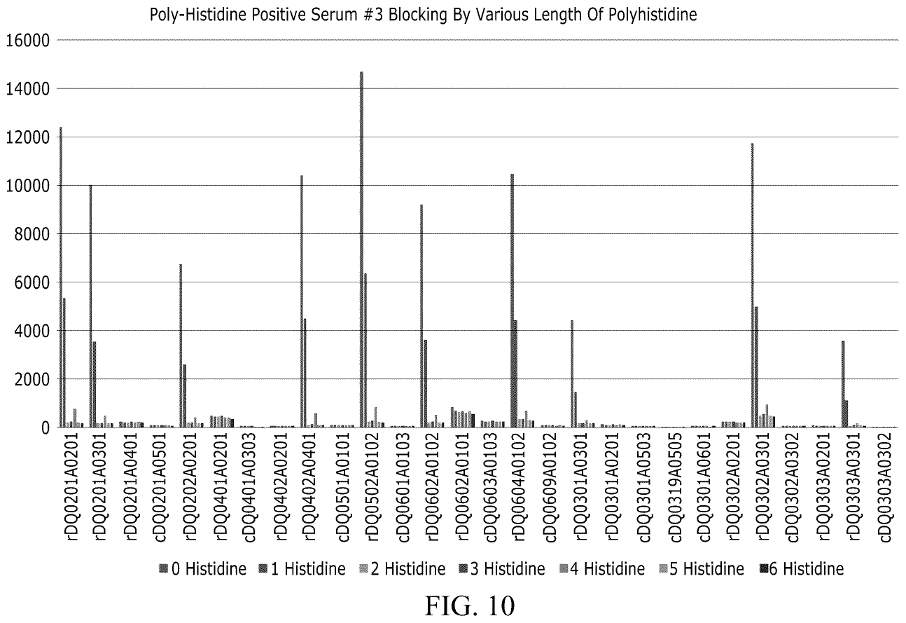

According to this example, five microgram of the DQA1 fusion proteins with different numbers of poly-histidine were used to block poly-histidine positive Serum No. 3 to determine the minimum number of poly-histidine involved in the epitope of poly-histidine positive Serum No. 3 antibody. According to the results shown in FIG. 10 a minimum of 2 histidine residues in the fusion protein are needed to block the anti-DQ specific poly-histidine antibody reactivity poly-histidine positive Serum No. 3. The possible epitope for the DQA1 specific antibody involves in part with the DQA1 specific sequence of SRHQ.

### Example 9

According to this example, poly-histidine positive Serum No. 4 was characterized Specifically, one microgram of each purified fusion protein from the following list was loaded on the 4-20% Tris-Glycine polyacrylamide gel and western blotted with poly-histidine positive serum No. 4.
1. GST-DRA1-LE-Hx6
2. GST-DQA1-LE-Hx6
3. GST-B-LE-Hx6
4. GST-A-LE-Hx6
5. GST-C-LE-Hx6
6. GST-C-LE-Hx5
7. GST-C-LE-Hx4
8. GST-C-LE-Hx3
9. GST-C-LE-Hx2
10. GST-C-LE-Hx1
11. GST-C-LE

According to these tests poly-histidine positive serum No. 4 reacts to only C-LE fusion proteins with at least 5 Histidine residues and the B-LE-Hx6. Since the carboxyl-end of the B and C antigens share the same amino acid A, the most likely epitope for the C-specific poly-histidine serum, poly-histidine serum No. 4, is ALEHHHHH (SEQ ID NO: 24).

### Example 10

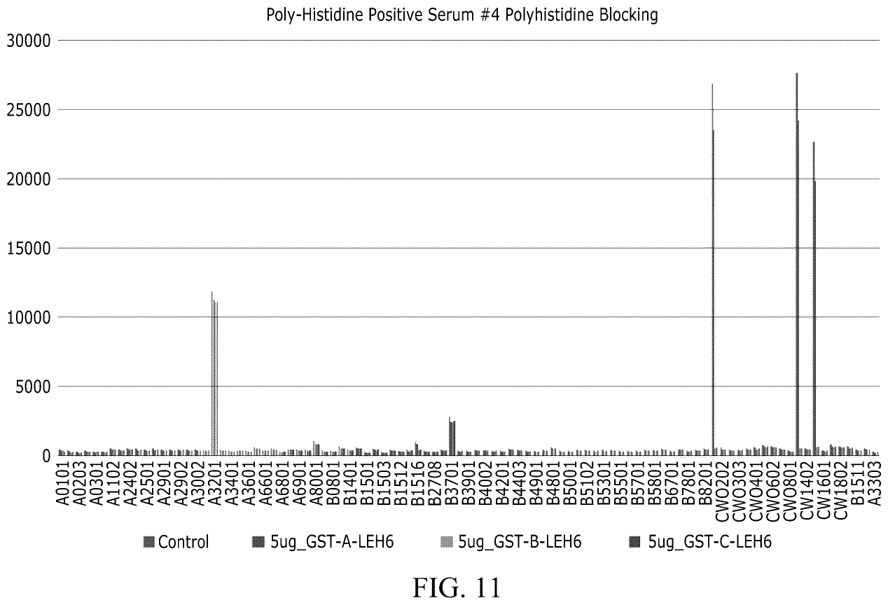

According to this example, twenty μl of poly-histidine positive serum No. 4 was preincubated with 5 μg of either GST-A-LE-Hx6, GST-B-LE-Hx6 and GST-C-LE-Hx6, then tested on LS1A04 Class I single antigen panel. The results presented in FIG. 11 showed GST-C-LE-Hx6 and GST-B-LE-Hx6 removed the antibody reactivity completely while GST-A-LE-Hx6 didn't remove the antibody reactivity.

### Example 11

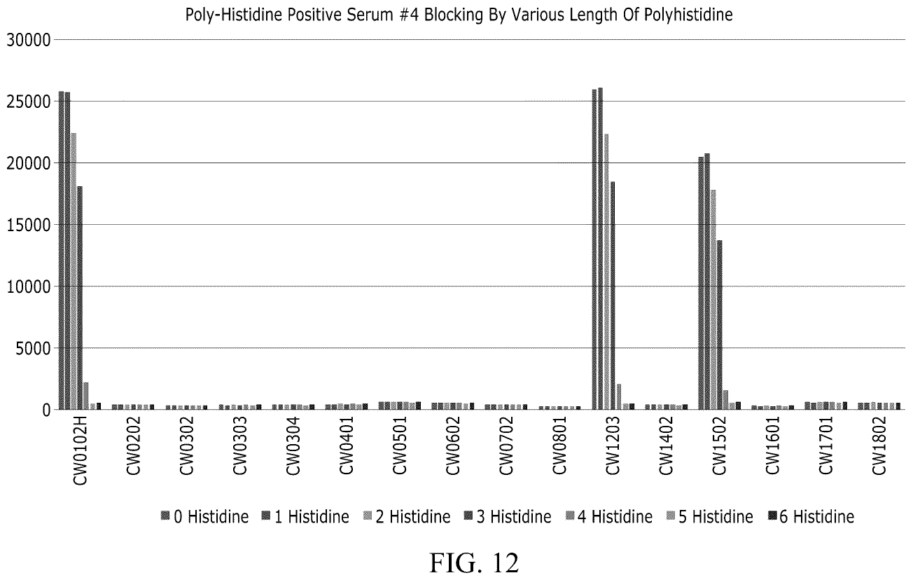

According to this example five micrograms of the C fusion proteins with different numbers of poly-histidine were used to block poly-histidine positive Serum No 4 to determine the minimum number of poly-histidine involved in the epitope poly-histidine positive Serum No 4 antibody. According to the results shown in FIG. 12 below a minimum of 5 Histidine in the fusion protein is needed to block the anti-C specific poly-histidine antibody reactivity by poly-histidine positive Serum No 4.

### Example 12

According to this example, 5 mg of Dynabeads® M-280 Tosyl activated beads (Catalog no. 14204) was washed and conjugated with 100 μg of the following poly-histidine containing proteins according to the manufacturer recommended protocol and resuspended in a final volume of 165 μl:
1. GST-DRA1-LE-Hx6
2. GST-DQA1-LE-Hx6
3. GST-C-LE-Hx6
4. GST-DRA1-LE-Hx6, GST-DQA1-LE-Hx6, GST-C-LE-Hx6 (33 μg each)
5. GST-DRA1-LE-Hx6, GST-DQA1-LE-Hx6, GST-C-LE-Hx6, Polyhistidine peptide (25 μg each)

Human sera were absorbed with beads conjugated with different protein mixtures as follows:
1. 20 μl of bead suspension were first pelleted in microcentrifuge tubes
2. Supernatant was removed
3. 20 μl serum (treated with 1 ul of 0.2M EDTA) was added to the pelleted beads, vortexed to resuspend the beads
4. Incubate at room temperature for 10 minutes.
5. Centrifuge to pellet the beads.
6. Serum (supernatant) is carefully removed from the tube for the assay.

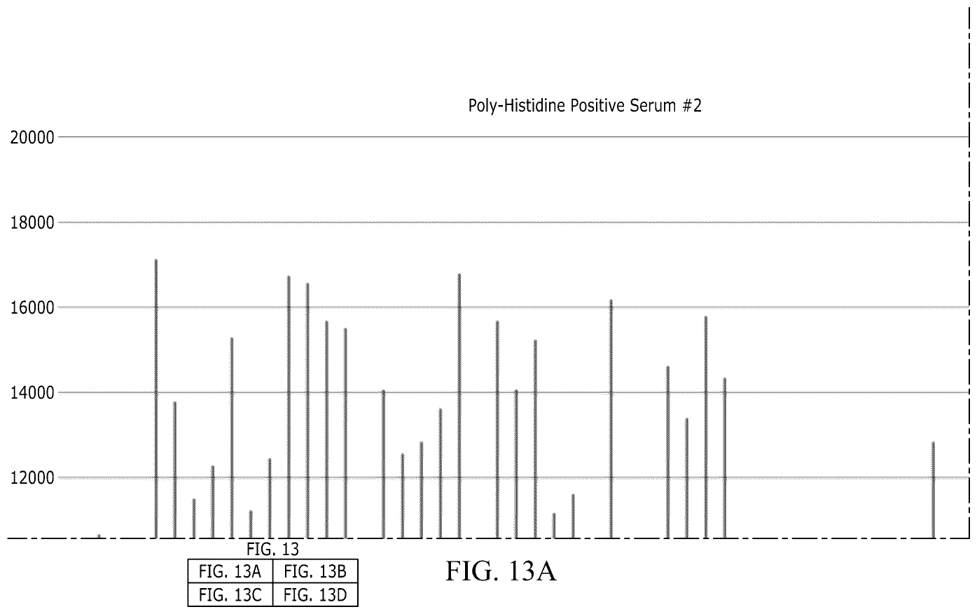

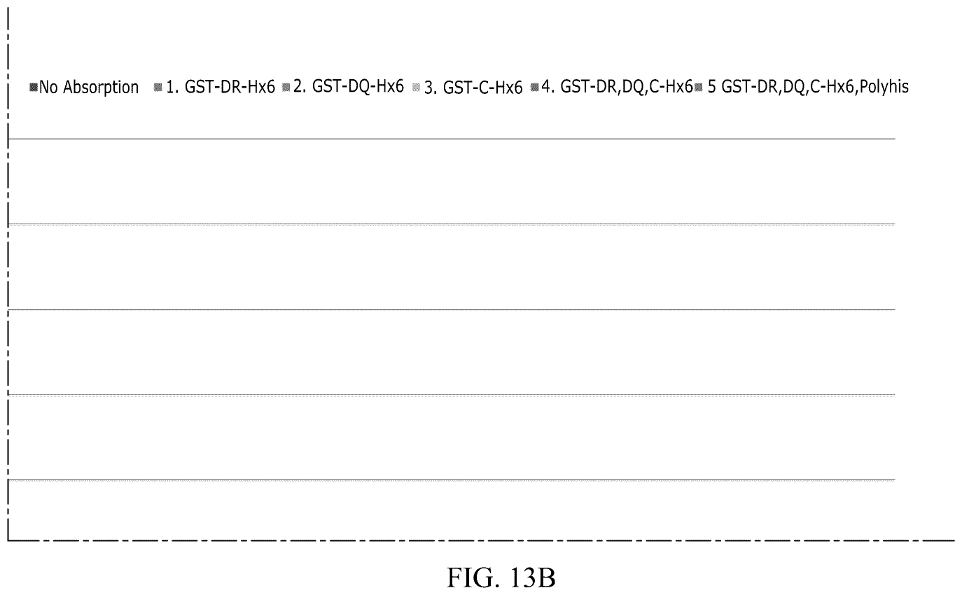

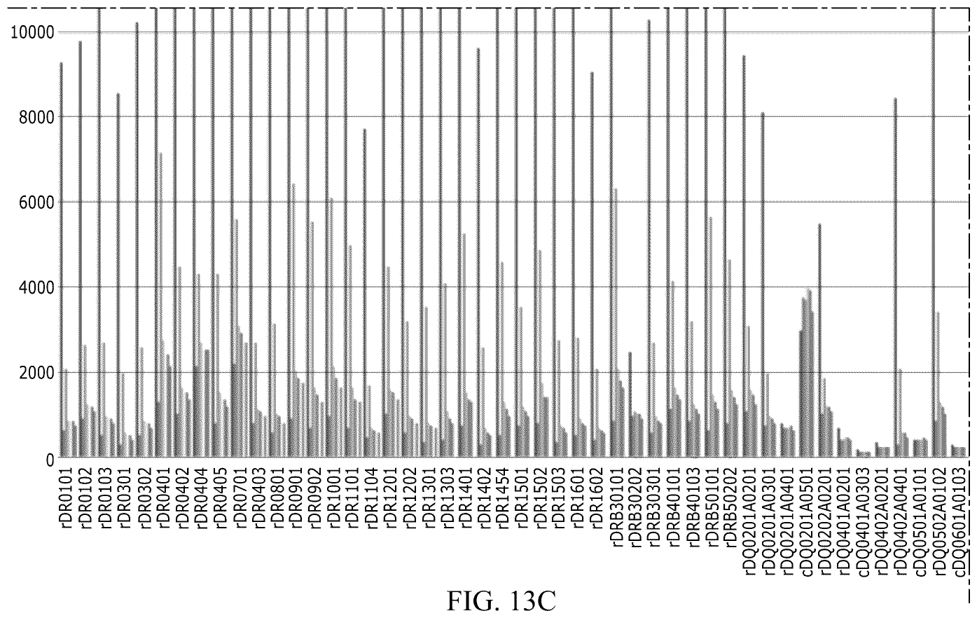

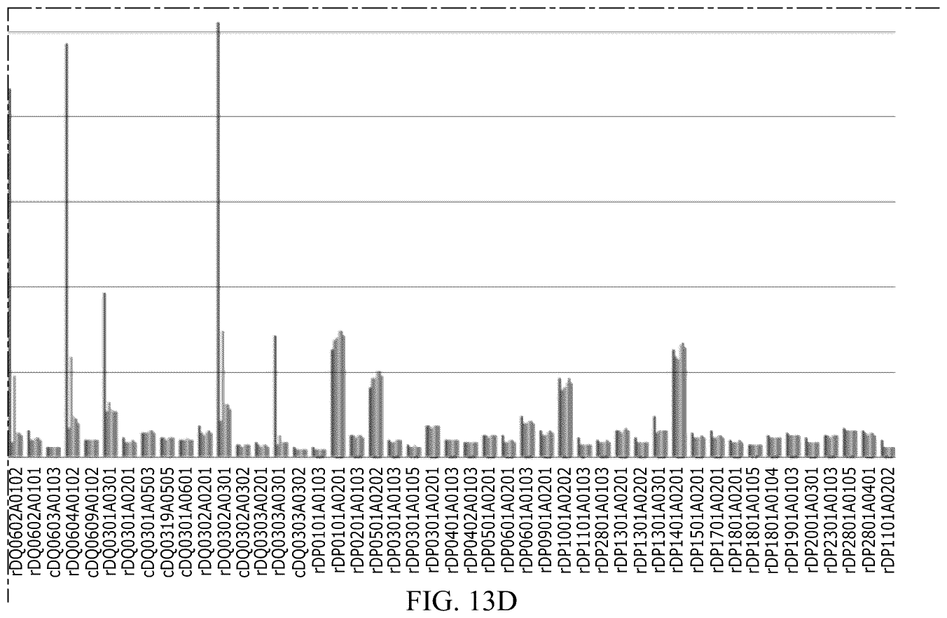

Poly-histidine serum No. 2 was absorbed with all 5 poly-histidine bead pools separately. As the results illustrated in FIG. 13 show, all the bead pools containing either poly-histidine peptide or fusion protein containing 6 histidine tag (Hx6) (#1-#5) were able to remove the poly-histidine antibody.

### Example 13

According to this example, DQA1 poly-histidine positive serum No. 4 was absorbed with all 5 poly-histidine bead pools separately. According to the results shown on the Table 4 below, only the bead pools containing GST-DQA1-LE-Hx6 (#2, #4 and #5) were able to remove the DQA1 specific poly-histidine antibody.

TABLE 4

| | rDQ0201 | rDQ0201 | rDQ0202 | rDQ0402 | rDQ0502 | rDQ0602 | rDQ0604 | rDQ0301 | rDQ0302 | rDQ0303 |
|---|---|---|---|---|---|---|---|---|---|---|
| Sample | A0201 | A0301 | A0201 | A0401 | A0102 | A0102 | A0102 | A0301 | A0301 | A0301 |
| No Absorption | 13054 | 10569 | 7405 | 11322 | 15259 | 10100 | 11107 | 4723 | 12493 | 4043 |
| 1. GST-DR-Hx6 | 13396 | 10997 | 7436 | 11542 | 14932 | 10077 | 11133 | 5006 | 12502 | 4161 |
| 2. GST-DQ-Hx6 | 294 | 240 | 256 | 193 | 328 | 332 | 416 | 275 | 609 | 152 |
| 3. GST-C-Hx6 | 12980 | 10850 | 7373 | 10632 | 15513 | 9965 | 11397 | 4666 | 12122 | 3924 |
| 4. GST-DR, | 336 | 263 | 285 | 211 | 360 | 329 | 440 | 283 | 674 | 165 |
| DQ, C-Hx6 | | | | | | | | | | |
| 5. GST-DR, | 401 | 300 | 294 | 245 | 405 | 346 | 481 | 283 | 686 | 166 |
| DQ, C-Hx6, polyhis | | | | | | | | | | |

HLA-C poly-histidine positive serum No. 4 was absorbed with all 5 poly-histidine bead pools separately. As the results shown in Table 5 below demonstrate, only the bead pools containing GST-C-LE-Hx6 (#3, #4 and #5) were able to remove the C specific poly-histidine antibody. An unrelated antibody that reacts to A3201 was not affected by any of the bead pools.

TABLE 5

| Sample | A3201 | CW0102H | CW1203 | CW1502 |
|---|---|---|---|---|
| No Absorption | 11511 | 24473 | 24856 | 20244 |
| 1. GST-DR-Hx6 | 11191 | 25083 | 25547 | 20636 |
| 2. GST-DQ-Hx6 | 11607 | 25456 | 25813 | 21033 |
| 3. GST-C-Hx6 | 12061 | 519 | 458 | 606 |
| 4. GST-DR, DQ, C-Hx6 | 11491 | 1005 | 943 | 938 |
| 5. GST-DR, DQ, C-Hx6, | 11737 | 768 | 700 | 804 |
| polyhis | | | | |

### Example 14

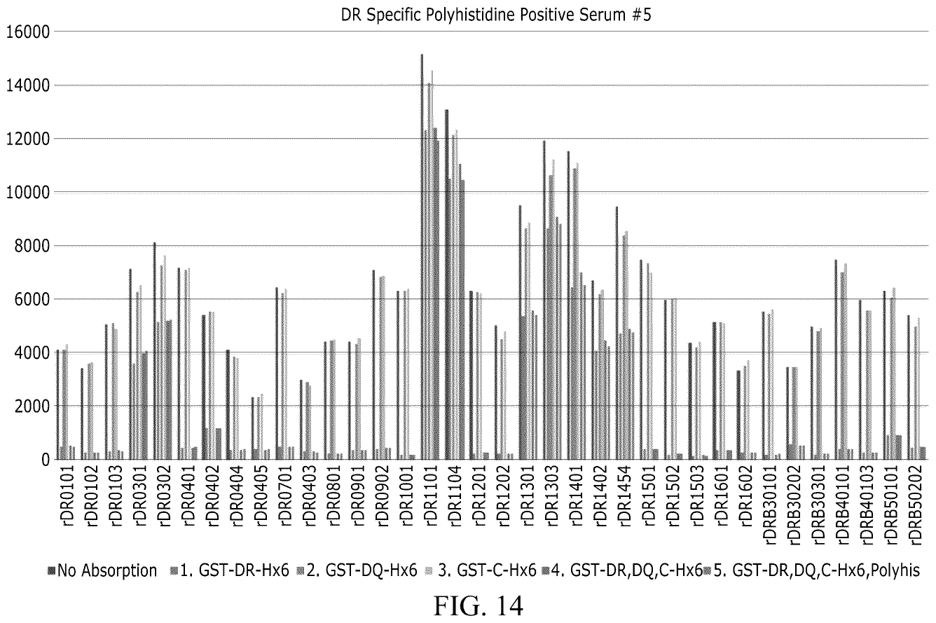

According to this example, DRA1 poly-histidine positive serum No. 5 was absorbed with all 5 poly-histidine bead pools separately. As the results shown in FIG. 14 illustrate, only the bead pools containing GST-DRA1-LE-Hx6 (#1, #4 and #5) were able to remove the DRA1 specific poly-histidine antibody.

Numerous modifications and variations in the practice of the invention are expected to occur to those skilled in the art upon consideration of the foregoing description on the presently preferred embodiments thereof. Consequently, the only limitations which should be placed upon the scope of the present invention are those that appear in the appended claims.

1. A method of screening a biological sample for antibodies that specifically bind a target antigen by:
obtaining a biological sample from a subject,
subjecting the biological sample to histidine polymers in order to block or remove anti-poly-histidine antibodies from the biological sample,
contacting the target antigen with the biological sample, and
detecting binding of antibodies in said biological sample to the target antigen.

2. The method of claim 1 wherein the target antigen is a synthetic antigen comprising a poly-histidine sequence of at least six contiguous histidine peptides.

3. The method of claim 2 wherein the target antigen is a recombinantly produced polypeptide including a poly-histidine sequence of at least six contiguous histidine peptides.

4. The method of claim 1 wherein the target antigen does not include a poly-histidine sequence of at least six contiguous histidine peptides.

5. The method of claim 2 wherein the recombinant poly-histidine sequence target antigen is produced by a mammalian, bacterial, yeast or insect expression system.

6. The method of claim 1 wherein the biological sample is obtained from a human subject.

7. The method of claim 1 wherein the biological sample is selected from the group consisting of blood, plasma and serum.

8. The method of claim 1 wherein the histidine polymers comprise at least two contiguous histidine peptides.

9. The method of claim 1 wherein the histidine polymers comprise at least six contiguous histidine peptides.

10. The method of claim 1 wherein the histidine polymer comprises a fusion protein of a recombinant protein with a poly-histidine sequence of at least two contiguous histidine peptides.

11. The method of claim 10 wherein the histidine polymer comprises a fusion protein comprising a C-terminal truncated version of the target antigen having at least two contiguous histidine peptides at its C-terminal end.

12. The method of claim 10 wherein the fusion protein comprises at least five contiguous histidine peptides.

13. The method of claim 1 wherein the target antigen is associated with a human or animal pathogen.

14. The method of claim 13 wherein the target antigen is associated with a pathogen selected from the group consisting of HIV, Lyme disease, syphilis, rotavirus and SARS associated coronavirus.

15. The method of claim 12 wherein the SARS associated coronavirus is SARS-CoV-2.

16. The method of claim 1 wherein the target antigen is a Human leukocyte antigen (HLA).

17. A method of screening for antibodies that specifically bind a target antigen comprising preparing a panel comprising a solid-phase substrate having said target antigen immobilized or attached thereto;
obtaining a biological sample from a subject,
subjecting the biological sample to histidine polymers to block or remove anti-poly-histidine antibodies from the serum sample,
contacting the panel with the biological sample, and
detecting binding of antibodies in said biological sample to the target antigen.

18. The method of claim 17 wherein the target antigen is a recombinantly produced polypeptide including a poly-histidine sequence of at least two contiguous histidine peptides.

19. The method of claim 17 wherein the target antigen is a recombinantly produced polypeptide including a poly-histidine sequence of at least six contiguous histidine peptides.

20. The method of claim 17 wherein the histidine polymer comprises a fusion protein of a recombinant protein with a poly-histidine sequence comprising a truncated version of a target antigen protein having at least two contiguous histidine peptides at its C-terminal.

21. The method of claim 20 wherein the anti-poly-histidine antibodies are removed from the serum sample by an affinity purification step in which they are contacted with histidine polymers immobilized on a solid substrate.

22. The method of claim 20 wherein the fusion protein comprises at least five contiguous histidine peptides.

23. The method of claim 17 wherein the target antigen is a synthetic antigen.

24. The method of claim 17 wherein the target antigen does not include a poly-histidine sequence of at least six contiguous histidine peptides.

25. The method of claim 17 wherein the biological sample is obtained from a human subject.

26. The method of claim 17 wherein the biological sample is selected from the group consisting of blood, plasma and serum.

27. The method of claim 17 wherein the histidine polymers comprise at least six contiguous histidine peptides.

28. The method of claim 17 wherein the target antigen is associated with a human or animal pathogen.

29. The method of claim 28 wherein the target antigen is associated with a pathogen selected from the group consisting of HIV, Lyme disease, syphilis, rotavirus and SARS associated coronavirus.

30. The method of claim 29 wherein the SARS associated coronavirus is SARS-CoV-2.

31. The method of claim 17 wherein the target antigen is a Human leukocyte antigen (HLA).

32. (canceled)

33. (canceled)

34. The method of claim 17 which is carried out on a lateral flow immunoassay device in which a sample fluid flows from a first position to a second position along a test strip and upon which the histidine polymers are immobilized between the first and second positions.

35. A method of screening for antibodies that specifically bind a target antigen comprising preparing a panel comprising a solid-phase substrate having a target antigen immobilized thereon and at least one other antigen selected from the group consisting of those consisting of antigens comprising at least two contiguous histidine peptides and fusion proteins with at least two contiguous histidine peptides and fusion peptides selected from the group of consisting of fusion proteins of a Human leukocyte antigen (HLA), and antigens selected from the group consisting of HIV, Lyme disease, syphilis, rotavirus and SARS associated coronavirus antigens with at least two contiguous histidine peptides,
obtaining a biological sample from a subject,
contacting the panel with the serum sample,
detecting binding of antibodies in said serum sample to the target antigen, and
detecting binding of antibodies in said serum sample to the poly-histidine antigen.

36. The method of claim 35 wherein the fusion protein comprises at least five contiguous histidine peptides.

37. A kit for screening for antibodies that specifically bind to a target antigen and/or to a poly-histidine antigen comprising a panel of solid-phase substrates wherein the panel comprises a solid phase substrate having a target antigen immobilized thereon and at least one other antigen selected from the group consisting of those comprising at least two contiguous histidine peptides, fusion proteins with at least two contiguous histidine peptides and fusion proteins of a Human leukocyte antigen (HLA), and antigens selected from the group consisting of HIV, Lyme disease, syphilis, rotavirus and SARS associated coronavirus antigens with at least two contiguous histidine peptides with at least 2 contiguous histidine peptides immobilized thereon.

38. The kit of claim 37 wherein the fusion protein comprises at least five contiguous histidine peptides.

39. A kit for removing antibodies that specifically bind to a poly-histidine antigen comprising a solid-phase substrate wherein an oligopeptide selected from the group consisting of those comprising at least two contiguous histidine peptides, fusion proteins with at least two contiguous histidine peptides and fusion proteins selected from the group of consisting fusion proteins of a Human leukocyte antigen (HLA), and antigens selected from the group consisting of HIV, Lyme disease, syphilis, rotavirus and SARS associated coronavirus antigens with at least 2 contiguous histidine peptides is immobilized thereon.

40. The kit of claim 39 wherein the fusion protein comprises at least 5 contiguous histidine peptides.

41. A kit comprising:
a solid phase substrate to which a target antigen is immobilized,
a solution comprising wherein an oligopeptide selected from the group consisting of those comprising at least two contiguous histidine peptides and fusion proteins selected from the group of consisting of fusion proteins of a Human leukocyte antigen (HLA), and antigens selected from the group consisting of HIV, Lyme disease, syphilis, rotavirus and SARS associated coronavirus antigens with at least two contiguous histidine peptides, and
a component capable of preventing non-specific antibody binding.

42. The kit of claim 40 wherein the fusion protein comprises at least 5 contiguous histidine peptides.

43. The kit of claim 40 wherein said target antigen is a recombinantly produced polypeptide including a poly-histidine sequence of at least six contiguous histidine peptides.

44. The kit of claim 40 wherein said target antigen does not include a poly-histidine sequence of at least six contiguous histidine peptides.

45. (canceled)

46. A lateral flow immunoassay device comprising a test strip having a first position at which a fluid sample is administered and a second position having a target antigen immobilized thereon upon which histidine polymers are immobilized between the first and second positions.


SEQUENCE LISTING (25 sequences)

SEQ ID NO: 1 - protein, 23 aa, Artificial Sequence
  Synthetic polypeptide
KGLRKSNAAERRGPLLEHHHHHH

SEQ ID NO: 2 - protein, 23 aa, Artificial Sequence
  Synthetic polypeptide
RGLRSVGASRHQGPLLEHHHHHH

SEQ ID NO: 3 - protein, 23 aa, Artificial Sequence
  Synthetic polypeptide
NSAQGSDESLIACKALEHHHHHH

SEQ ID NO: 4 - protein, 6 aa, Artificial Sequence
  Synthetic polypeptide
HHHHHH

SEQ ID NO: 5 - protein, 23 aa, Artificial Sequence
  Synthetic polypeptide
KGLRKSNAAERRGPLLEHHHHHH

SEQ ID NO: 6 - protein, 23 aa, Artificial Sequence
  Synthetic polypeptide
RGLRSVGASRHQGPLLEHHHHHH

SEQ ID NO: 7 - protein, 22 aa, Artificial Sequence
  Synthetic polypeptide
RGLRSVGASRHQGPLLEHHHHH

SEQ ID NO: 8 - protein, 21 aa, Artificial Sequence
  Synthetic polypeptide
RGLRSVGASRHQGPLLEHHHH

SEQ ID NO: 9 - protein, 20 aa, Artificial Sequence
  Synthetic polypeptide
RGLRSVGASRHQGPLLEHHH

SEQ ID NO: 10 - protein, 19 aa, Artificial Sequence
  Synthetic polypeptide
RGLRSVGASRHQGPLLEHH

SEQ ID NO: 11 - protein, 18 aa, Artificial Sequence
  Synthetic polypeptide
RGLRSVGASRHQGPLLEH

SEQ ID NO: 12 - protein, 17 aa, Artificial Sequence
  Synthetic polypeptide
RGLRSVGASRHQGPLLE

SEQ ID NO: 13 - protein, 15 aa, Artificial Sequence
  Synthetic polypeptide
RGLRSVGASRHQGPL

SEQ ID NO: 14 - protein, 15 aa, Artificial Sequence
  Synthetic polypeptide
VLTACKVLEHHHHHH

SEQ ID NO: 15 - protein, 12 aa, Artificial Sequence
  Synthetic polypeptide
VLTALEHHHHHH

SEQ ID NO: 16 - protein, 15 aa, Artificial Sequence
  Synthetic polypeptide
SLIACKALEHHHHHH

SEQ ID NO: 17 - protein, 14 aa, Artificial Sequence
  Synthetic polypeptide
SLIACKALEHHHHH

SEQ ID NO: 18 - protein, 13 aa, Artificial Sequence
  Synthetic polypeptide
SLIACKALEHHHH

SEQ ID NO: 19 - protein, 12 aa, Artificial Sequence
  Synthetic polypeptide
SLIACKALEHHH

SEQ ID NO: 20 - protein, 11 aa, Artificial Sequence
  Synthetic polypeptide
SLIACKALEHH

SEQ ID NO: 21 - protein, 10 aa, Artificial Sequence
  Synthetic polypeptide
SLIACKALEH

SEQ ID NO: 22 - protein, 9 aa, Artificial Sequence
  Synthetic polypeptide
SLIACKALE

SEQ ID NO: 23 - protein, 7 aa, Artificial Sequence
  Synthetic polypeptide
SLIACKA

SEQ ID NO: 24 - protein, 8 aa, Artificial Sequence
  Synthetic polypeptide
ALEHHHHH

SEQ ID NO: 25 - protein, 21 aa, Artificial Sequence
  Synthetic polypeptide
SDGGGGSGGGGSGGGGSGGGG